SEQUENCE LISTING (32 sequences)

SEQ ID NO: 1 - DNA, 1219 bp, synthetic construct
  source 1..1219: mol_type = other DNA
gggaaataagagagaaaagaagagtaagaagaaatataagagccaccatgaaatgggtgaccttcatcagcctgctgtttctgttcagctctgcctacagcgtgttcaccctggaagatttcgtgggcgactggagacaaaccgccggctacaacctggaccaggtgctggaacagggcggcgtctcctccctgttccagaacctgggcgttagcgtcacccctatccagagaatcgtgctgagcggcgagaacggcctgaagatcgatatccacgtgatcatcccctacgagggactgtctggcgatcagatgggccagatcgagaagattttcaaggtggtgtatcctgtggacgaccaccacttcaaagtgatcctgcactacggcacactggtgatcgatggcgtcacaccaaacatgatcgactacttcggcagaccttacgagggcatcgccgtgtttgacggcaagaagattacagtgacaggcaccctgtggaacggcaacaagatcatcgatgagagactgatcaaccccgacggctctctgctgtttagagtgaccatcaatggagtgacaggatggcggctgtgcgaaagaatcctggctggcggaggcggcagcggcggaggcggaagcgacagctggatggaagaggtgatcaagctgtgtggcagggaactggtgcggcagatcgccatctgcggaatgagcacctggagcaagagatctctcagccaggaggacgcccctcagacccctcggcctgtggccgagatcgtgcccagcttcatcaacaaggacacccaactctactccgccctggctaataaatgctgccatgtgggttgtaccaagcggagcctggccagattctgctgataagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 2 - DNA, 47 bp, synthetic construct
  source 1..47: mol_type = other DNA
gggaaataagagagaaaagaagagtaagaagaaatataagagccacc

SEQ ID NO: 3 - DNA, 54 bp, synthetic construct
  source 1..54: mol_type = other DNA
atgaaatgggtgaccttcatcagcctgctgtttctgttcagctctgcctacagc

SEQ ID NO: 4 - DNA, 510 bp, synthetic construct
  source 1..510: mol_type = other DNA
gtgttcaccctggaagatttcgtgggcgactggagacaaaccgccggctacaacctggaccaggtgctggaacagggcggcgtctcctccctgttccagaacctgggcgttagcgtcacccctatccagagaatcgtgctgagcggcgagaacggcctgaagatcgatatccacgtgatcatcccctacgagggactgtctggcgatcagatgggccagatcgagaagattttcaaggtggtgtatcctgtggacgaccaccacttcaaagtgatcctgcactacggcacactggtgatcgatggcgtcacaccaaacatgatcgactacttcggcagaccttacgagggcatcgccgtgtttgacggcaagaagattacagtgacaggcaccctgtggaacggcaacaagatcatcgatgagagactgatcaaccccgacggctctctgctgtttagagtgaccatcaatggagtgacaggatggcggctgtgcgaaagaatcctggct

SEQ ID NO: 5 - DNA, 30 bp, synthetic construct
  source 1..30: mol_type = other DNA
ggcggaggcggcagcggcggaggcggaagc

SEQ ID NO: 6 - DNA, 84 bp, synthetic construct
  source 1..84: mol_type = other DNA
gacagctggatggaagaggtgatcaagctgtgtggcagggaactggtgcggcagatcgccatctgcggaatgagcacctggagc

SEQ ID NO: 7 - DNA, 84 bp, synthetic construct
  source 1..84: mol_type = other DNA
aagagatctctcagccaggaggacgcccctcagacccctcggcctgtggccgagatcgtgcccagcttcatcaacaaggacacc

SEQ ID NO: 8 - DNA, 72 bp, synthetic construct
  source 1..72: mol_type = other DNA
caactctactccgccctggctaataaatgctgccatgtgggttgtaccaagcggagcctggccagattctgc

SEQ ID NO: 9 - DNA, 132 bp, synthetic construct
  source 1..132: mol_type = other DNA
gctcgctttcttgctgtccaatttctattaaaggttcctttgttccctaagtccaactactaaactgggggatattatgaagggccttgagcatctggattctgcctaataaaaaacatttattttcattgc

SEQ ID NO: 10 - DNA, 200 bp, synthetic construct
  source 1..200: mol_type = other DNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 11 - protein, 278 aa, synthetic construct
MKWVTFISLLFLFSSAYSVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAGGGGSGGGGSDSWMEEVIKLCGRELVRQIAICGMSTWSKRSLSQEDAPQTPRPVAEIVPSFINKDTQLYSALANKCCHVGCTKRSLARFC

SEQ ID NO: 12 - protein, 18 aa, synthetic construct
MKWVTFISLLFLFSSAYS

SEQ ID NO: 13 - protein, 170 aa, synthetic construct
VFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 14 - protein, 10 aa, synthetic construct
GGGGSGGGGS

SEQ ID NO: 15 - protein, 28 aa, synthetic construct
DSWMEEVIKLCGRELVRQIAICGMSTWS

SEQ ID NO: 16 - protein, 28 aa, synthetic construct
KRSLSQEDAPQTPRPVAEIVPSFINKDT

SEQ ID NO: 17 - protein, 24 aa, synthetic construct
QLYSALANKCCHVGCTKRSLARFC

SEQ ID NO: 18 - DNA, 673 bp, synthetic construct
  source 1..673: mol_type = other DNA
gggaaataagagagaaaagaagagtaagaagaaatataagagccaccatgaaatgggtcacattcatcagcctgctgtttctgttcagcagcgcctactctgatagctggatggaagaagtgatcaagctgtgtggaagagagctggtgcggcaaatcgccatctgcggcatgagcacctggtccaagagatctctgagccaggaggacgcccctcagacccctagaccagtggccgagatcgtgcccagcttcattaacaaggacacccagctgtactccgctctggccaacaagtgctgccacgtgggctgtacaaagcggagcctcgctagattctgcgctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 19 - DNA, 54 bp, synthetic construct
  source 1..54: mol_type = other DNA
atgaaatgggtcacattcatcagcctgctgtttctgttcagcagcgcctactct

SEQ ID NO: 20 - DNA, 84 bp, synthetic construct
  source 1..84: mol_type = other DNA
gatagctggatggaagaagtgatcaagctgtgtggaagagagctggtgcggcaaatcgccatctgcggcatgagcacctggtcc

SEQ ID NO: 21 - DNA, 84 bp, synthetic construct
  source 1..84: mol_type = other DNA
aagagatctctgagccaggaggacgcccctcagacccctagaccagtggccgagatcgtgcccagcttcattaacaaggacacc

SEQ ID NO: 22 - DNA, 72 bp, synthetic construct
  source 1..72: mol_type = other DNA
cagctgtactccgctctggccaacaagtgctgccacgtgggctgtacaaagcggagcctcgctagattctgc

SEQ ID NO: 23 - protein, 98 aa, synthetic construct
MKWVTFISLLFLFSSAYSDSWMEEVIKLCGRELVRQIAICGMSTWSKRSLSQEDAPQTPRPVAEIVPSFINKDTQLYSALANKCCHVGCTKRSLARFC

SEQ ID NO: 24 - DNA, 1050 bp, synthetic construct
  source 1..1050: mol_type = other DNA
gggaaataagagagaaaagaagagtaagaagaaatataagagccaccatgaaatgggtcacctttatcagcctgctgttcctgttcagcagcgcctacagcgtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgggaggcgggggcagccacgagaccacccccaacaaggggagcgggaccacgtccggcacaactagactgctttccggccatacatgctttacacttactgggctgctggggactcttgtaactatggggctcctcacatgataagctgccttctgcggggcttgccttctggccatgcccttcttctctcccttgcacctgtacctcttggtctttgaataaagcctgagtaggaaggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 25 - DNA, 54 bp, synthetic construct
  source 1..54: mol_type = other DNA
atgaaatgggtcacctttatcagcctgctgttcctgttcagcagcgcctacagc

SEQ ID NO: 26 - DNA, 510 bp, synthetic construct
  source 1..510: mol_type = other DNA
gtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcg

SEQ ID NO: 27 - DNA, 15 bp, synthetic construct
  source 1..15: mol_type = other DNA
ggaggcgggggcagc

SEQ ID NO: 28 - DNA, 123 bp, synthetic construct
  source 1..123: mol_type = other DNA
cacgagaccacccccaacaaggggagcgggaccacgtccggcacaactagactgctttccggccatacatgctttacacttactgggctgctggggactcttgtaactatggggctcctcaca

SEQ ID NO: 29 - DNA, 95 bp, synthetic construct
  source 1..95: mol_type = other DNA
gctgccttctgcggggcttgccttctggccatgcccttcttctctcccttgcacctgtacctcttggtctttgaataaagcctgagtaggaaggc

SEQ ID NO: 30 - protein, 234 aa, synthetic construct
MKWVTFISLLFLFSSAYSVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAGGGGSHETTPNKGSGTTSGTTRLLSGHTCFTLTGLLGTLVTMGLLT

SEQ ID NO: 31 - protein, 5 aa, synthetic construct
GGGGS

SEQ ID NO: 32 - protein, 41 aa, synthetic construct
HETTPNKGSGTTSGTTRLLSGHTCFTLTGLLGTLVTMGLLT

(19) United States
(12) Patent Application Publication
(10) Pub. No.: US 2025/0042967 A1
(43) Pub. Date: Feb. 6, 2025

(54) mRNA TREATMENT TO INDUCE EXPRESSION OF RELAXIN FOR REPRODUCTIVE APPLICATIONS

(71) Applicants: Peter Ryan, Starkville, MS (US); Phillip J Santangelo, Atlanta, GA (US); Amelia Woolums, Starkville, MS (US); Elmer Heath King, Starkville, MS (US); Merrilee Thoresen, Starkville, MS (US); Daryll Vanover, Atlanta, GA (US); Federico Hoffmann, Starkville, MS (US)

(72) Inventors: Peter Ryan, Starkville, MS (US); Phillip J Santangelo, Atlanta, GA (US); Amelia Woolums, Starkville, MS (US); Elmer Heath King, Starkville, MS (US); Merrilee Thoresen, Starkville, MS (US); Daryll Vanover, Atlanta, GA (US); Federico Hoffmann, Starkville, MS (US)

(21) Appl. No.: 18/794,438

(22) Filed: Aug. 5, 2024

Related U.S. Application Data
(60) Provisional application No. 63/517,521, filed on Aug. 3, 2023.

(51) Int. Cl. C07K 14/64; A61K 38/00; A61P 15/04

(52) U.S. Cl. CPC C07K 14/64; A61P 15/04; A61K 38/00

(57) ABSTRACT

Synthetic H2 RLN mRNA constructs that induce expression of relaxin, methods of preparing such synthetic H2 RLN mRNA constructs, and methods of administering such constructs to mammals to facilitate delivery of offspring or methods of administering such constructs to non-pregnant mammals to facilitate procedures involving the reproductive tract.

### CROSS-REFERENCE TO RELATED APPLICATIONS

This application claims the benefit of U.S. provisional application No. 63/517,521, filed on Aug. 3, 2023, the disclosure of which is hereby incorporated by reference in its entirety.

### STATEMENT OF GOVERNMENT SUPPORT

This invention was made with government support under grant/contract no. NIH R01AI155007 awarded by the National Institutes of Health (NIH), National Institute of Allergy and Infectious Diseases (NIAID). The Government has certain rights in the invention.

### INCORPORATION OF MATERIAL OF XML SEQUENCE LISTING BY REFERENCE

The sequence listing submitted herewith as an XML file named “MSU-1016USSequenceListing” created on Aug. 2, 2024 and 34,000 bytes in size, is hereby incorporated by reference in its entirety.

### FIELD OF THE INVENTION

The present invention relates to the field of facilitating reproduction in humans and animals, especially cattle. The present invention can be employed to reduce or prevent difficult deliveries of offspring in female cattle and other mammals, including humans.

### BACKGROUND OF THE INVENTION

Female cattle and other animals, including humans, sometimes have trouble delivering their young (dystocia) because of inadequate relaxation or size of the mother's reproductive tract. Such situations can lead to difficult deliveries that may be harmful to the mother and/or the offspring. In some cases, surgery may be required to deliver the offspring, which poses additional risks to the mother and the offspring.

Relaxin (RLN) is a reproductive hormone that enhances connective tissue remodeling during pregnancy and parturition and relaxin deficiencies have led to prolonged delivery, increased rate of stillbirths, and incomplete softening of connective tissues in some species (i.e. pigs and rodents).

Production of the hormone relaxin by the mother's reproductive tissues helps with relaxation of the reproductive tract and thereby facilitates delivery of the offspring. However, some females do not produce adequate relaxin. Moreover, many female cattle have lost the ability to naturally produce relaxin, but it has been found that such cattle can have the desired response, relaxation of the reproductive tract, if relaxin is administered by an exogenous source.

Relaxin was discovered in 1926. See “Experimental relaxation of the pubic ligament of the guinea pig,” Hisaw, F. L. (1926). Experimental relaxation of the pubic ligament of the guinea pig. Proceedings of the Society for Experimental Biology and Medicine, 23 (8), 661-663.

Relaxin is known for connective tissue remodeling during pregnancy and parturition. At times female individuals do not produce adequate relaxin, which can lead to difficult delivery. Cattle, in particular, have lost the ability to produce relaxin, but they can respond if given relaxin. Females sometimes have trouble delivering their babies, and treatment with relaxin could help prevent this.

Many cattle are incapable of relaxin synthesis but maintain a functional relaxin receptor, RXFP1. Malone, L., Opazo, J. C., Ryan, P. L., & Hoffmann, F. G. (2017). “Progressive erosion of the Relaxin1 gene in bovids”, General and Comparative Endocrinology, 252, 12-17 and Dai, Y., Ivell, R., Liu, X., Janowski, D., & Anand-Ivell, R. (2017), “Relaxin-family peptide receptors 1 and 2 are fully functional in the bovine”, Frontiers in Physiology. 8:359.

Previous attempts were made to elicit relaxin effects in order to reduce the incidence of dystocia but were unsuccessful. Dystocia affects almost 70% of all dairy operations (NAHMS 2014). Dystocia Increases the risk of mastitis, metritis, and retained placentae. Lombard, J. E., Garry, F. B., Tomlinson, S. M., & Garber, L. P. (2007). “Impacts of dystocia on health and survival of dairy calves”, Journal of Dairy Science, 90 (4), 1751-1760. Treatment of sequelae is estimated to cost 4× more than dystocia. Oltenacu, P. A., Frick, A., & Lindhe, B. (1988). “Use of statistical modelling and decision analysis to estimate financial losses due to dystocia and other diseases in Swedish cattle”, Veterinarian Scandinavia Supplementum. 84, 353-355.

Relaxin hormone therapy has demonstrated some efficacy to reduce dystocia in dairy heifers. However, the results are variable and depend on the mode of administration, the method of hormone preparation: porcine vs. recombinant human relaxin. It has been found that, in vitro, human relaxin is superior to porcine relaxin for activating bovine receptors (Dai et al., 2017).

More specifically, in cattle, attempts using purified porcine or recombinant human RLN to reduce the incidence of dystocia in heifers presented variable results. However, Human 2 (H2) RLN has a high affinity for the bovine RLN receptor (RXFP1).

It has also been attempted to deliver relaxin hormone exogenously. However, it is difficult to deliver relaxin hormone to the site where it needs to work, and relaxin does not last for very long when given exogenously.

As such, there is a need to improve upon the administration of relaxin to cattle to reduce the incidence of dystocia. This and other objectives of the invention are discussed in the summary and detailed descriptions below.

### SUMMARY OF THE INVENTION

The present invention relates to combinations of H2-RLN with mRNA therapeutics. The present invention also relates to H2 RLN mRNA therapy for dystocia of mammals, including cattle and humans.

In one embodiment, the present invention provides a synthetic H2 RLN mRNA construct that induces expression of relaxin with a secretion signal, optionally in combination with a reporter protein.

In some embodiments, the foregoing synthetic H2 RLN mRNA construct is encoded by a nucleotide sequence selected from the sequences of SEQ ID NO: 1 and SEQ ID NO: 18.

In some embodiments, the foregoing synthetic H2 RLN mRNA construct encodes an amino acid sequence selected from the sequences of SEQ ID NO: 11 and SEQ ID NO: 23.

In some embodiments, the present invention provides a synthetic H2 RLN-mRNA construct that induces expression of relaxin with a secretion signal.

In another embodiment, the present invention provides a method for facilitating delivery of offspring in mammals including a step of administering a synthetic H2 RLN mRNA construct of any of the foregoing embodiments to a mammal prior to delivery of the offspring.

In some embodiments of the above method, the mammal is a human.

In some embodiments of the above method, the mammal is a bovine.

In any one of the embodiments of the above method, the synthetic H2 RLN-mRNA construct is administered to the kidney.

In any one of the embodiments of the above method, the synthetic H2 RLN-mRNA construct is administered to the epithelial cells.

In any one of the embodiments of the above method, a dose of 0.5 mg-10 mg of the synthetic H2 RLN-mRNA construct is administered.

In any one of the embodiments of the above method, two or more doses of 0.5 mg-10 mg of the synthetic H2 RLNmRNA construct are administered.

### BRIEF DESCRIPTION OF THE DRAWINGS

FIG. 1 shows the cell culture samples and collection used in Example 1.

FIG. 2 shows an ELISA assay for detection of H2 Relaxin.

FIG. 3 shows an mRNA construct encoding Relaxin.

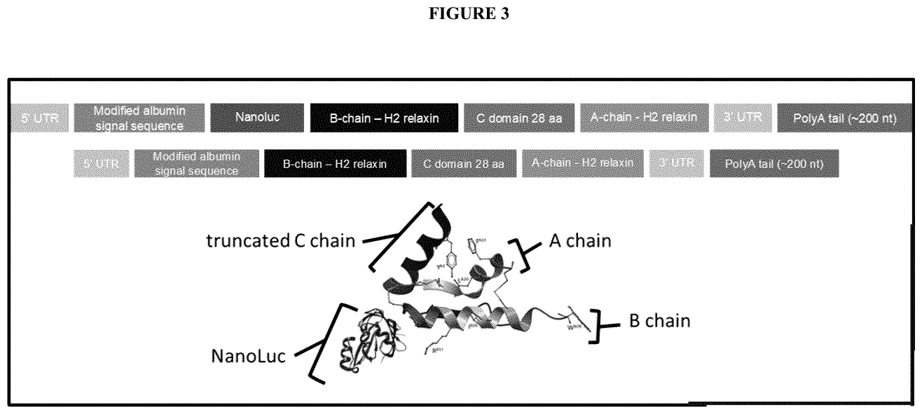

FIG. 4 shows Relaxin-NanoLuc fusion protein expression in BK cell lysates at 3, 6, 9, 12, 24 and 48 hours post-transfection.

FIG. 5 shows Relaxin-NanoLuc fusion protein expression in BVEC cell lysates at 3, 6, 9, 12, 24 and 48 hours post-transfection.

FIG. 6 shows the secretion of Relaxin-NanoLuc fusion proteins in BK supernatants at 3, 6, 9, 12, 24 and 48 hours post-transfection.

FIG. 7 shows the secretion of Relaxin-NanoLuc fusion proteins in BVEC supernatants at 3, 6, 9, 12, 24 and 48 hours post-transfection.

FIG. 8 shows the concentration of H2 Relaxin in BK cell lysates of at 3, 6, 9, 12, 24 and 48 hours post-transfection.

FIG. 9 shows the concentration of H2 Relaxin in BK cell supernatants at 3, 6, 9, 12, 24 and 48 hours post-transfection.

FIG. 10 shows that bovine reproductive mucosa is receptive to transfection, resulting in high levels of expression at the ectocervix (rhe target tissue for Relaxin treatment).

FIG. 11 shows the detected levels of expression of H2-Relaxin in vaginal, cervical, and uterine tissues.

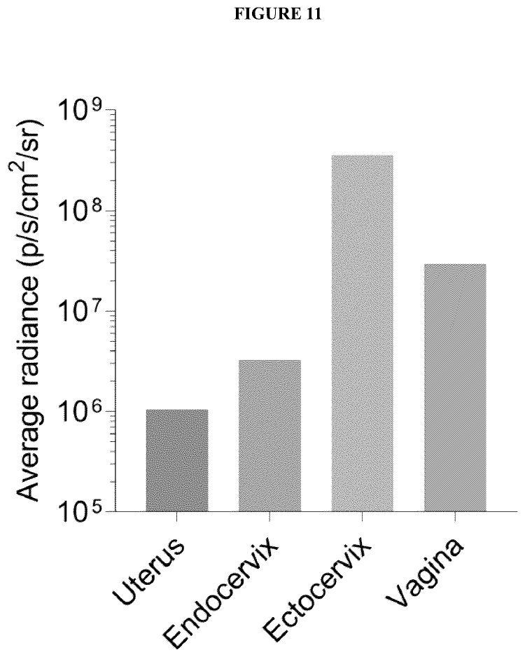

FIGS. 12A-12F show histologic sections of the uterine endometrium from control untreated cows (FIGS. 12A, 12B, and 12C) and treated cows (FIGS. 12D, 12E, and 12F).

### DETAILED DESCRIPTION OF THE INVENTION

In one aspect the present invention relates to a H2 RLN mRNA construct that will induce production of relaxin when administered to a mammal. Skilled persons are capable of making such constructs since the amino acid sequence of relaxin is in the public domain and standard techniques for making mRNA constructs for targeting a particular amino acid sequence such as that of relaxin are known in the art.

In some embodiments, the H2 RLN mRNA construct may comprise a secretion signal-encoding region (e.g., a secretion signal-encoding region that allows an encoded target entity or entities to be secreted upon translation by cells). In some embodiments, such a secretion signal-encoding region may be or comprise a non-human secretion signal. In some embodiments, such a secretion signal-encoding region may be or comprise a human secretion signal.

In some embodiments, the H2 RLN mRNA may comprise at least one non-coding sequence element. Examples of non-coding sequence elements include but are not limited to a 3′ untranslated region (UTR), a 5′ UTR, a cap structure for co-transcriptional capping of mRNA, a poly adenine (polyA) tail, and any combination thereof.

In some embodiments, the H2 RLN mRNA may comprise the above elements/region in combination with a region encoding a reporter protein (such as NanoLuciferase) or a marker protein.

In another aspect, the invention relates to a method for making the H2 RLN mRNA construct of the invention.

In one aspect, the invention relates to inducing relaxin production rapidly and exactly where it is needed by applying messenger RNA (mRNA) directly to the surface of the cells of the reproductive tract. Treatment of cells of the female reproductive tract of both cattle and humans with messenger RNA (mRNA) encoding relaxin can induce the cells to produce relaxin. Thus, it has been demonstrated that administration of mRNA to the female reproductive tract has the potential to cause relaxin release exactly where it is needed and when it is needed. It is also possible to modify the mRNA component to prolong relaxin production.

Suitable dosages of the mRNA construct are from 0.5 mg-10 mg per dose, or 1.0 mg to 7 mg per dose, or 1.0 mg to 5 mg per dose or about 2 mg to 4 mg per dose, or about 2 mg per dose. These dosages are particularly suitable for bovines.

Dosing can be carried out over a period of 1-20 days. One to five doses of the amounts given above can be administered over this period of 1-20 days, or over 1 to 10 days, or over 2 to 5 days or at days 1 and 3. A preferred dosing regimen for bovines is 2-3 mg doses at 0 and 48 hours. Dosing should take place 1-20 days prior to the expected date of delivery, or 1-20 days prior or 1 to 5 days prior or 1 to 2 days prior to the expected date of delivery.

Use of mRNA will provide better controlled and more localized production of relaxin than has been possible with prior efforts that rely on administration of the hormone. Thus it is possible that treatment of the reproductive tract of cattle, women, or other female individuals with mRNA encoding relaxin could ease delivery in cases where delivery of the offspring is impeded due to inadequate relaxation.

The relaxin encoding mRNA construct of the invention may be administered to pregnant mammals that can express relaxin, including but not limited to human, bovine, equine, porcine, canine, feline, and domestic or zoological cetacean species. In one aspect, the invention could be used to prevent difficult deliveries of offspring in female cattle and other mammals as discussed herein. In addition, the mRNA construct can also be administered to non-pregnant mammals that can express relaxin, including but not limited to human, bovine, equine, porcine, canine, feline, and domestic or zoological cetacean species. For example, the mRNA construct may be administered to facilitate dilation of the cervix for therapeutic procedures where dilation and curettage is needed.

In one embodiment, the relaxin encoding mRNA may be formulated in a pharmaceutical composition. As used herein, the term “pharmaceutical composition” includes the relaxin encoding mRNA of the present invention as an active agent with one or more pharmaceutically acceptable carriers or excipients. In some embodiments, the active agent is present in a unit dose amount appropriate for administration in a therapeutic regimen that shows a statistically significant probability of achieving a predetermined therapeutic effect when administered to a relevant population (such as the mammalian species described herein).

In some embodiments, the pharmaceutical compositions may be specially formulated for administration in solid form, in liquid form, in lipid nanoparticles (LNP), or in polymeric nanoparticles for application or administration by, including, but not limited to, the following:
aerosol administration (e.g., trans cervically or topically to the reproductive mucosal surface), systemic absorption, boluses, powders, granules, pastes for application to the reproductive tract or reproductive mucosal surface;
parenteral administration, for example, by subcutaneous, intramuscular, intravenous or tissue injection as, for example, a sterile solution or suspension, or sustained-release formulation;
topical application, for example, as a cream, ointment, patch, or a controlled-release patch or spray applied to the skin, reproductive tract, or reproductive mucosal surface;
intravaginal (e.g., by incorporation into thin polymer films for intravaginal delivery) or intrarectal administration, for example, as a pessary (e.g., by polymeric rings for insertion around the cervix allowing for slow release of the relaxin encoding mRNA), cream, or foam; and transdermal administration (e.g., by a microneedle patch or microarray patch).

For instance, in the Examples provided below, relaxin encoding mRNA was formulated in nuclease free water and sprayed onto the surface of the cervical epithelium using a Teleflex MADgic Mucosal Atomization device. In addition, the relaxin encoding mRNA may be formulated in lipid nanoparticles (LNP) or in polymeric nanoparticles and applied in a similar fashion. Exemplary mRNA constructs are shown in Tables 1-2 below.

TABLE 1

| | Nucleic Acid Sequence | Amino Acid Sequence |
|---|---|---|
| Name | SEQ ID NO: | SEQ ID NO: |
| Nluc-H2 Relaxin | SEQ ID NO: 1 | SEQ ID NO: 11 |
| H2 Relaxin | SEQ ID NO: 18 | SEQ ID NO: 23 |
| Anchored NanoLuc | SEQ ID NO: 24 | SEQ ID NO: 30 |

Nluc-H2 Relaxin (SEQ ID NO: 1) consists of, starting from the 5′ end to the 3′ end: 5′UTR (SEQ ID NO: 2), a signal sequence (SEQ ID NO: 3), NanoLuciferase (SEQ ID NO: 4), a GS linker (SEQ ID NO: 5), a B-chain (SEQ ID NO: 6), a C-domain (SEQ ID NO: 7), an A-chain-H2 relaxin (SEQ ID NO: 8), stop codons (TGATAA), 3′UTR (SEQ ID NO: 9), and a PolyA tail (SEQ ID NO: 10).

Nluc-H2 Relaxin (SEQ ID NO: 11) consists of a signal sequence (SEQ ID NO: 12), NanoLuciferase (SEQ ID NO: 13), a GS linker (SEQ ID NO: 14), a B-chain (SEQ ID NO: 15), a C-domain (SEQ ID NO: 16), and an A-chain-H2 relaxin (SEQ ID NO: 17).

H2 Relaxin (SEQ ID NO: 18) consists of, starting from the 5′end to the 3′end: 5′UTR (SEQ ID NO: 2), a signal sequence (SEQ ID NO: 19), a B-chain (SEQ ID NO: 20), a C-domain (SEQ ID NO: 21), an A-chain-H2 relaxin (SEQ ID NO: 22), stop codons (TGATAA), 3′UTR (SEQ ID NO: 9), and a PolyA tail (SEQ ID NO: 10).

H2 relaxin (SEQ ID NO: 23) consists of a signal sequence (SEQ ID NO: 12), a B-chain (SEQ ID NO: 15), a C-domain (SEQ ID NO: 16), and an A-chain-H2 relaxin (SEQ ID NO: 17).

Anchored NanoLuc (SEQ ID NO: 24) consists of, starting from the 5′end to the 3′end: 5′UTR (SEQ ID NO: 2), a signal sequence (SEQ ID NO: 25), NanoLuciferase (SEQ ID NO: 26), a GS linker (SEQ ID NO: 27), a human DAF GPI anchor (SEQ ID NO: 28), stop codons (TGATAA), 3′UTR (SEQ ID NO: 29), and a PolyA tail (SEQ ID NO: 10).

Anchored NanoLuc (SEQ ID NO: 30) consists of a signal sequence (SEQ ID NO: 12), NanoLuciferase (SEQ ID NO: 13), a GS linker (SEQ ID NO: 31), and a human DAF GPI anchor (SEQ ID NO: 32).

TABLE 2

| | | | | | | | A-Chain- | Human | | | |
|---|---|---|---|---|---|---|---|---|---|---|---|
| | | | | GS | | | 2H | DAF GPI | Stop | | |
| Name | 5′UTR | Signal Sequence | NanoLuc | linker | B-chain | C-domain | relaxin | anchor | codons | 3′UTR | PolyAT |
| Nluc-H2 | GGGAA | ATGAAATGGGTG | GTGTTCACCCTGGAAGATTT | GGCGGAG | GACAGCTGG | AAGAGATC | CAACTCTA | | TGATAA | GCTCGCTTT | AAAAAAAA |
| Relaxin | ATAAGA | ACCTTCATGAGC | CGTGGGCGACTGGAGACA | GCGGCAG | ATGGAAGAG | TCTCAGCG | CTCGGCCG | | | CTTGCTGTC | AAAAAAAA |
| (nucleic | GAGAA | CTGCTGTTTCTGT | AACCGCCGGCTACAACCT | CGGCGGA | GTGATCAAG | AGGAGGAG | TGGCTAATA | | | CAATTTCTAT | AAAAAAAA |
| acid | AAGAA | TCAGCTCTGCCT | GGACCAGGTGCTGGAACA | GGGGGAA | CTGTGTGGC | GCCCCTCA | AATGCTGC | | | TAAAGGTTC | AAAAAAAA |
| sequence) | GAGTAA | ACAGC | GGGCGGCGTCTCCTCCCT | GC | AGGGAACTG | GACCCCTC | CATGTGGG | | | CTTTGTTCCC | AAAAAAAA |
| (SEQ ID | GAAGA | (SEQ ID NO: 3) | GTTCCAGAACCTGGGCGTT | (SEQ ID | GTGCGGCA | GGCCTGTG | TTGTACCAA | | | TAAGTCCAA | AAAAAAAA |
| NO: 1) | AATATA | | AGCGTCACCCCTATCCAGA | NO: 5) | GATCGCCAT | GCCGAGAT | GCGGAGC | | | CTACTAAACT | AAAAAAAA |
| | AGAGC | | GAATCGTGCTGAGCGGCG | | CTGCGGAAT | CGTGCCCA | CTGGCCAG | | | GGGGGATAT | AAAAAAAA |
| | CACC | | AGAACGGCCTGAAGATCGA | | GAGCACCTG | GCTTCATCA | ATTCTGC | | | TATGAAGGG | AAAAAAAA |
| | (SEQ ID | | TATCCACGTGATCATCCCC | | GAGC | ACAAGGAC | (SEQ ID | | | CCTTGAGCA | AAAAAAAA |
| | NO: 2) | | TAGGAGGGACTGTCTGGGG | | (SEQ ID | ACC | NO: 8) | | | TCTGGATTCT | AAAAAAAA |
| | | | ATCAGATGGGCCAGATCGA | | NO: 6) | (SEQ ID | | | | GCCTAATAA | AAAAAAAA |
| | | | GAAGATTTTCAAGGTGGTGT | | | NO: 7) | | | | AAAACATTTA | AAAAAAAA |
| | | | ATCCTGTGGACGACCACCA | | | | | | | TTTTCATTGC | AAAAAAAA |
| | | | CTTCAAAGTGATCCTGCACT | | | | | | | (SEQ ID | AAAAAAAA |
| | | | ACGGCACACTGGTGATCGA | | | | | | | NO: 9) | AAAAAAAA |
| | | | TGGCGTCACACCAAACATG | | | | | | | | AAAAAAAA |
| | | | ATCGACTACTTCGGCAGAC | | | | | | | | AAAAAAAA |
| | | | CTTAGGAGGGCATCGCCGT | | | | | | | | AAAAAAAA |
| | | | GTTTGACGGCAAGAAGATT | | | | | | | | AAAAAAAA |
| | | | ACAGTGACAGGCACCCTGT | | | | | | | | AAAAAAAA |
| | | | GGAACGGCAACAAGATCAT | | | | | | | | AAAAAAAA |
| | | | CGATGAGAGACTGATCAAC | | | | | | | | AAAAAAAA |
| | | | CCCGACGGCTCTCTGCTGT | | | | | | | | AAAAAAAA |
| | | | TTAGAGTGACCATCAATGGA | | | | | | | | AAAAAAAA |
| | | | GTGACAGGATGGCGGCTGT | | | | | | | | (SEQ ID |
| | | | GCGAAAGAATCCTGGCT | | | | | | | | NO: 10) |
| | | | (SEQ ID NO: 4) | | | | | | | | |
| Nluc-H2 | | MKWVTFISLLFLF | VFTLEDFVGDWRQTAGYNL | GGGGSGG | DSWMEEVIK | KRSLSQEDA | QLYSALANK | | | | |
| relaxin | | SSAYS | DQVLEQGGVSSLFQNLGVS | GGS | LCGRELVRQ | PQTPRPVAE | CCHVGCTK | | | | |
| (amino | | (SEQ ID NO: 12) | VTPIQRIVLSGENGLKIDIH | (SEQ ID | IAICGMSTW | IVPSFINKD | RSLARFC | | | | |
| acid | | | VIIPYEGLSGDQMGQIEKIF | NO: 14) | S | T | (SEQ ID | | | | |
| sequence) | | | KVVYPVDDHHFKVILHYGTL | | (SEQ ID | (SEQ ID | NO: 17) | | | | |
| (SEQ ID | | | VIDGVTPNMIDYFGRPYEGI | | NO: 15) | NO: 16) | | | | | |
| NO: 11) | | | AVFDGKKITVTGTLWNGNKI | | | | | | | | |
| | | | IDERLINPDGSLLFRVTING | | | | | | | | |
| | | | VTGWRLCERILA | | | | | | | | |
| | | | (SEQ ID NO: 13) | | | | | | | | |
| H2 | SEQ ID | ATGAAATGGGTC | | | GATAGCTGG | AAGAGATC | CAGCTGTA | | TGATAA | SEQ ID | SEQ ID |
| Relaxin | NO: 2 | ACATTCATCAGC | | | ATGGAAGAA | TCTGAGCC | CTCCGCTC | | | NO: 9 | NO: 10 |
| (nucleic | | CTGCTGTTTCTGT | | | GTGATCAAG | AGGAGGAC | TGGCCAAC | | | | |
| acid | | TCAGCAGCGCC | | | CTGTGTGGA | GCCCCTCA | AAGTGCTG | | | | |
| sequence) | | TACTCT | | | AGAGAGCTG | GACCCCTA | CCACGTGG | | | | |
| (SEQ ID | | (SEQ ID NO: 19) | | | GTCGGGCAA | GACCAGTG | GCTGTACA | | | | |
| NO: 18) | | | | | ATCGCCATC | GCCGAGAT | AAGCGGAG | | | | |
| | | | | | TGCGGCATG | CGTGCCCA | CCTCGCTA | | | | |
| | | | | | AGCACCTGG | GCTTCATTA | GATTCTGC | | | | |
| | | | | | TCC | ACAAGGAC | (SEQ ID | | | | |
| | | | | | (SEQ ID | ACC | NO: 22) | | | | |
| | | | | | NO: 20) | (SEQ ID | | | | | |
| | | | | | | NO: 21) | | | | | |
| H2 | | SEQ ID NO: 12 | | | SEQ ID | SEQ ID | SEQ ID | | | | |
| Relaxin | | | | | NO: 15 | NO: 16 | NO: 17) | | | | |
| (amino | | | | | | | | | | | |
| acid | | | | | | | | | | | |
| sequence) | | | | | | | | | | | |
| (SEQ ID | | | | | | | | | | | |
| NO: 23) | | | | | | | | | | | |
| Anchored | SEQ ID | ATGAAATGGGTC | ATTGTCCTGAGCGGTGAAA | GGAGGCG | | | | CACGAGACC | | GCTGCCTTC | SEQ ID |
| Nanoluc | NO: 2 | ACCTTTATCAGC | ATGGGCTGAAGATCGACAT | GGGGCAG | | | | ACCCCCAAC | | TGCGGGCT | NO: 10 |
| (nucleic | | CTGCTGTTCCTG | CCATGTCATCATCCCGTAT | C | | | | AAGGGGAG | | TGCCTTCTG | |
| acid | | TTCAGCAGCGC | GAAGGTCTGAGCGGCGAC | (SEQ ID | | | | CGGGACCA | | GCCATGCCC | |
| sequence) | | CTACAGC | CAAATGGGCCAGATCGAAA | NO: 27) | | | | CGTCCGGC | | TTCTTCTCTC | |
| (SEQ ID | | (SEQ ID NO: 25) | AAATTTTTAAGGTGGTGTAC | | | | | ACAACTAGA | | CCTTGCACC | |
| NO: 24) | | | CCTGTGGATGATCATCACTT | | | | | CTGCTTTCC | | TGTACCTCTT | |
| | | | TAAGGTGATCCTGCACTATG | | | | | GGCCATACA | | GGTCTTTGAA | |
| | | | GGACACTGGTAATCGACGG | | | | | TGCTTTACA | | TAAAGCCTG | |
| | | | GGTTACGCCGAACATGATC | | | | | CTTACTGGG | | AGTAGGAAG | |
| | | | GACTATTTCGGACGGCCGT | | | | | CTGCTGGGG | | GC | |
| | | | ATGAAGGCATCGCCGTGTT | | | | | ACTCTTGTA | | (SEQ ID | |
| | | | CGACGGCAAAAAGATCACT | | | | | ACTATGGGG | | NO: 29) | |
| | | | GTAACAGGGACCCTGTGGA | | | | | CTCCTCACA | | | |
| | | | ACGGCAACAAAATTATCGA | | | | | (SEQ ID | | | |
| | | | CGAGCGCCTGATCAACCC | | | | | NO: 28) | | | |
| | | | CGACGGCTCCCTGCTGTTC | | | | | | | | |
| | | | CGAGTAACCATCAACGGAG | | | | | | | | |
| | | | TGACCGGCTGGCGGCTGT | | | | | | | | |
| | | | GCGAACGCATTCTGGCG | | | | | | | | |
| | | | (SEQ ID NO: 26) | | | | | | | | |

| | | | | |
|---|---|---|---|---|
| Anchored | SEQ ID NO: 12 | SEQ ID NO: 13 | GGGGS | HETTPNKGS |
| NanoLuc | | | (SEQ ID | GTTSGTTRL |
| (amino | | | NO: 31) | LSGHTCFTL |
| sequence) | | | | TGLLGTLVT |
| SEQ ID | | | | MGLLT |
| NO: 30 | | | | (SEQ ID |
| | | | | NO: 32) |

### Example 1. Method for Making Relaxin Encoding mRNA

The relaxin encoding mRNA of the present invention was prepared by IVT (in vitro transcription). IVT is a well-known procedure in the art that allows template-directed synthesis of RNA molecules of any sequence from short oligonucleotides to several kilobases. See Beckert, Bertrand & Masquida, Benoit. (2011). Synthesis of RNA by In Vitro Transcription. In: Methods in Molecular Biology (Clifton, NJ), vol 703, pages 29-41, 10.1007/978-1-59745-248-9_3.; the disclosure of which is incorporated herein in its entirety.

For IVT, plasmids encoding the relaxin peptides were linearized with Not-I HF (New England Biolabs) overnight at 37° C. Linearized templates were purified by sodium acetate (Thermo Fisher Scientific) precipitation and rehydrated with nuclease-free water. IVT was performed overnight at 37° C. using the HiScribe T7 Kit (NEB) following the manufacturer's instructions (N1-methyl-pseudouridine modified). The resulting RNA was treated with DNase I (Aldevron) for 30 min to remove the template and was then purified using lithium chloride precipitation (Thermo Fisher Scientific). The RNA was heat denatured at 65° C. for 10 min before capping with a type 1 cap structure using guanylyl transferase and 2′-O-methyltransferase (Aldevron). mRNA was then purified by lithium chloride precipitation, treated with alkaline phosphatase (NEB) and purified again. mRNA concentration was measured using a Nanodrop. Purified mRNA products were analyzed by gel electrophoresis (Agilent Fragment Analyzer) to ensure purity.

### Example 2

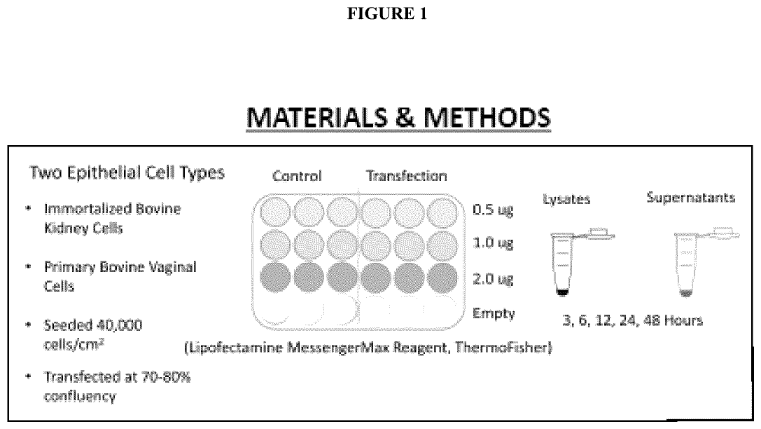

Bovine kidney (BK) and primary bovine epithelial cells (BVEC) were transfected with a synthetic H2 RLN-NanoLuciferase (NanoLuc) mRNA construct with a secretion signal. The bovine kidney (BK) and primary bovine epithelial cells (BVEC) were transfected with 0.5, 1 or 2 μg synthetic mRNA. At 3, 6, 12, 24 and 48 hours post-transfection, cell lysates and supernatants were collected for detection of H2 RLN. The cell culture samples and collection are shown in FIG. 1.

### H2 Relaxin ELISA Assay

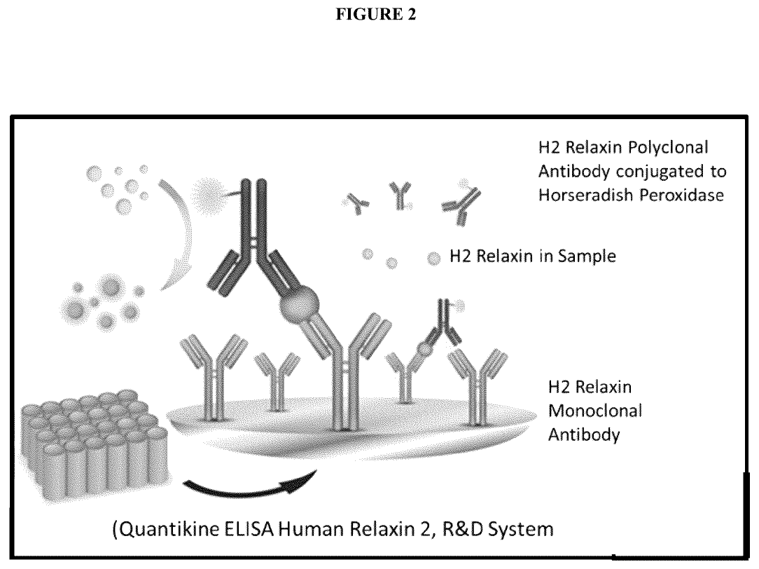

Detection was carried out indirectly via Nano-Glo Assay® (Promega) or directly via ELISA (R&D Systems) as shown in FIG. 2. Luminescence demonstrated that BVEC expressed H2 RLN in cell lysates for all observed time points with a decline only being observed at 48 hours in the BVE cells. Cell supernatants exhibited increasing levels of luminescence, indicating secretion of H2 RLN.

### Results

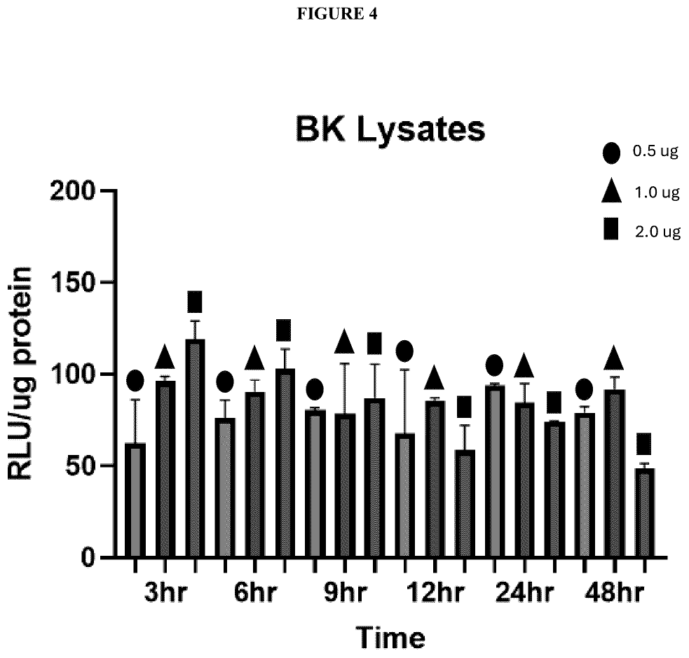

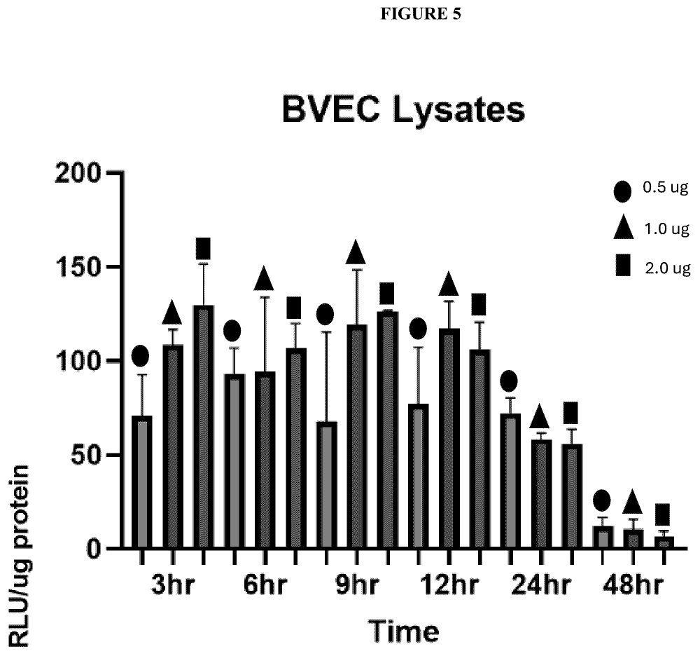

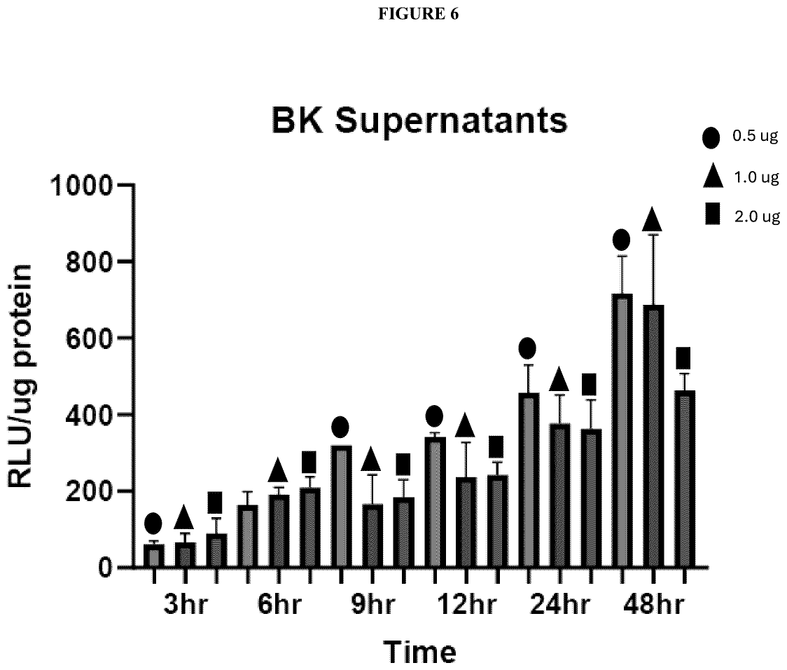

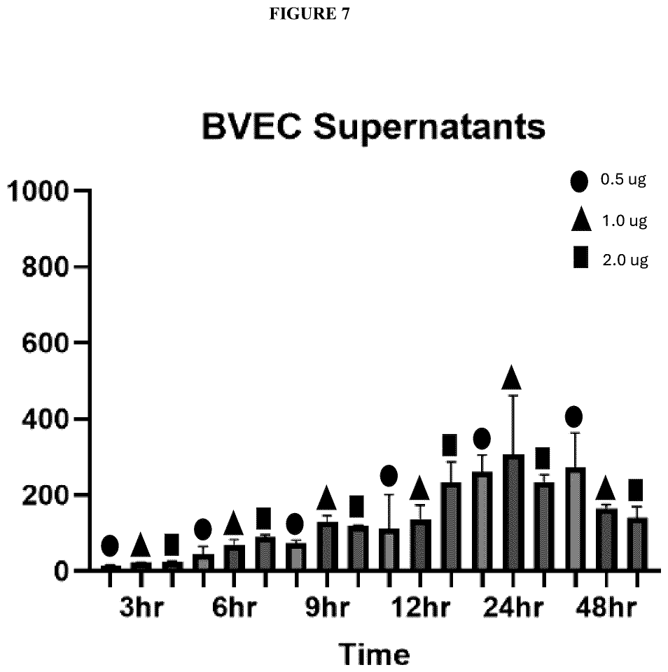

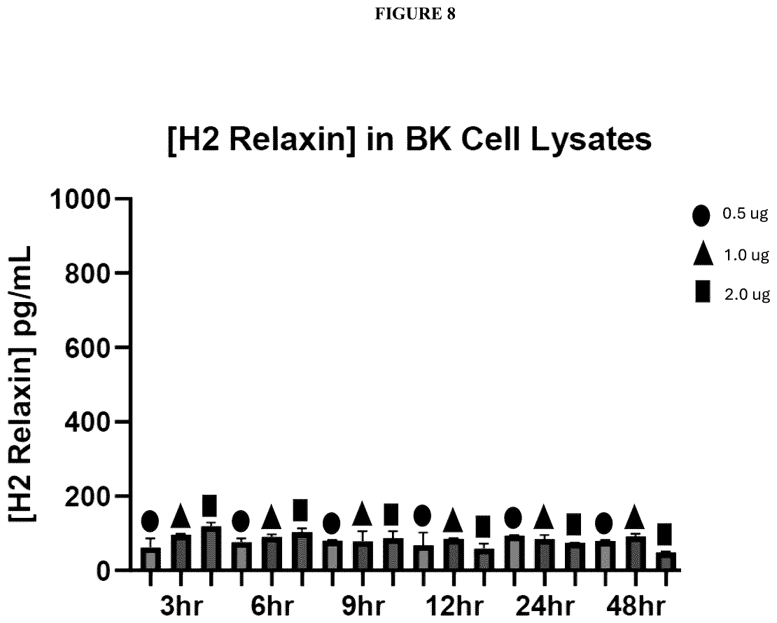

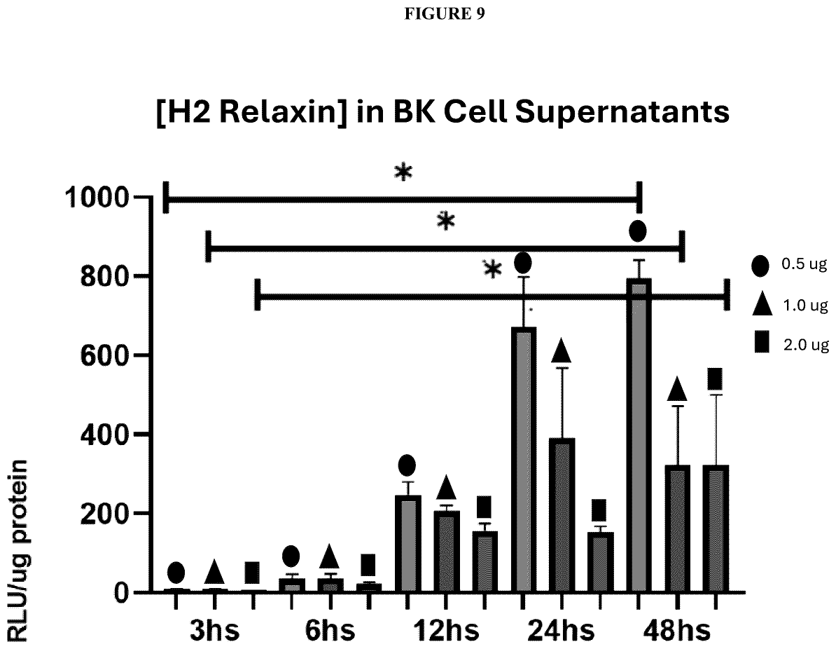

Bovine epithelial cells transfected with synthetic mRNA expressed relaxin. Luminescence demonstrated relaxin-NanoLuc fusion protein expression in cell lysates at all observed time points, with a decline only at 48 hours in the BVEC cells (FIGS. 4 and 5). Increasing luminescence was shown in supernatants over time, indicating secretion of relaxin-NanoLuc fusion protein (FIGS. 6 and 7). FIGS. 4, 5, and 8 show similar temporal trends for NanoGlo and ELISA assays. Increasing concentrations of H2 RLN were observed from 6-48 hours in the supernatants from the BK cells transfected with the lowest concentration of mRNA (0.5 μg). The lowest mRNA concentration (0.5 μg) induced the greatest expression (FIG. 9).

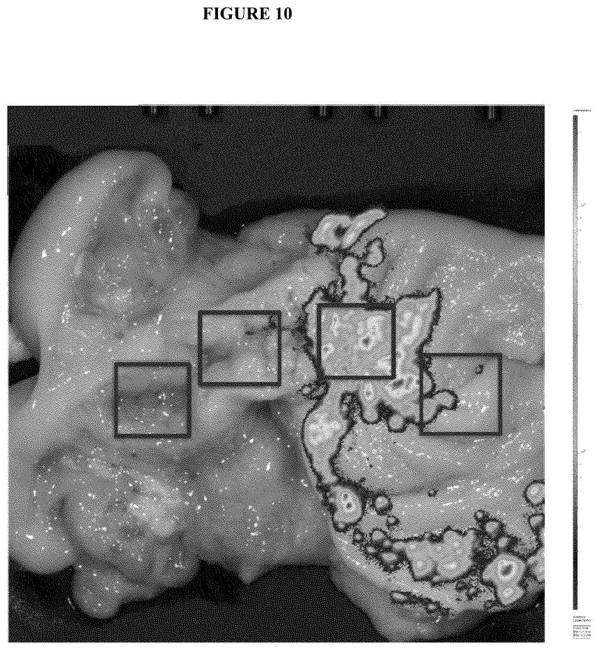

Furthermore, the in vivo transfection of a 6-month-old dairy heifer with NanoLucmRNA demonstrated that the bovine reproductive mucosa is receptive to transfection resulting in high levels of expression at the ectocervix, the target tissue for H2 RLN (FIGS. 9 and 10). This data provides evidence supporting the effectiveness of in vivo transfections with H₂ RLN mRNA as a novel approach in reducing dystocia in heifers.

### Example 3

Transfection of the bovine female reproductive tract was investigated in vivo. Nonpregnant cull dairy cows (n=2) were examined and confirmed to be in normal general and reproductive health prior to intravaginal treatment with mRNA encoding H2 RLN at time 0 and 48 hours. Vaginal secretions were collected over the course of 120 hrs post-transfection. Reproductive tissues were harvested at 120 hours for detection of H2 RLN directly via western blot. Detectable concentrations of H2 RLN were present in samples of vaginal, cervical, and uterine tissues from both treated animals.

### Objectives

1. To deliver synthetic mRNA encoding human relaxin (H2 relaxin) to bovine vaginal and cervical mucosa, and to compare concentrations of relaxin in vaginal secretions of treated cows to concentrations in secretions of control cows treated with noncoding mRNA.
2. To assess the effect of mRNA treatment on physical and histological characteristics of the reproductive tract of treated cows and compare to controls cows treated with noncoding mRNA.

### Methods

Animals: Estimate of age, weight, color, breed, and any other relevant information were recorded.

### Prior to Study:

Cows were palpated and treated with GnRH and progesterone via controlled internal drug release (CIDR) 35 days prior to the study start date. After 14 days, the CIDR was removed, and prostaglandin injection was administered. Cows underwent reproductive evaluation approximately 5 days prior to the study and any cows exhibiting abnormal cyclicity or follicular development were removed from the study to achieve n=6. Cows selected to proceed were transported to the research pen. Two days prior to treatment another injection of prostaglandin was administered, and the study began 48 hours afterward.
Day −35 prior: GnRH Injection and CIDR Placement (Saturday, April 15^{th}).
Day −21 prior: Removal of CIDR and prostaglandin injection (Saturday, April 29^{th}).
Day −5 prior: Evaluation of Ovaries. Move cows to CVM (Monday, May 15^{th}).
Day −2 prior: Prostaglandin Inj. (Thursday, May 18^{th}).

### Treatment of Cows:

Nonpregnant cull dairy cows (n=6) were examined to confirm general and reproductive health, including a rectal examination and uterine ultrasound to confirm absence of gross uterine pathology, and a vaginal speculum examination to confirm absence of gross vaginal or cervical pathology. Cows confirmed to be in normal general and reproductive health were randomly assigned to be treated with mRNA encoding H2 relaxin (n=3 cows) or water only (n=3 cows) at times 0 and 48 hours. mRNA treatment (2 mg per dose) was delivered in water by spray applied through a vaginal speculum to the cervical and proximal vaginal mucosa.

### Sample Collection:

Blood was collected into a serum tube and serum was separated and aliquoted for determination of progesterone level and systemic relaxin levels on Day 0 (prior to treatment), Day 2 prior to the 2^{nd} treatment and on day 5.

After the 120 hours of sampling, cows were euthanized and the reproductive tracts were dissected out; sections of the vulva, vagina, cervix, uterus, and ovaries were removed and duplicate sections were fixed in formalin for histopathologic analysis or snap frozen in liquid nitrogen for detection of relaxin in homogenized tissues by ELISA. Sections fixed in formalin were stained with H&E and Masson's Trichrome and assessed by a pathologist unaware of the treatment status of each cow to provide semi-quantitative scoring of connective tissue reorganization indicating effects of relaxin.

TABLE 3

| Day, Time | Study | | Vaginal | Blood | Ultrasound |
|---|---|---|---|---|---|
| (hour) | 2 | Timeline | Swabs | Collection | Exams |
| Treatment | 20 | 7-8 am | *prior to | * | |
| 1 = D0, T0 | May | | treatment | | |
| T6 | | 1 pm | | | |
| T12 | | 7 pm | | | |
| D1, T24 | | 7 am | * | | |
| Treatment | 22 | 7-8 am | *prior to | * | |
| 2 = D2, | May | | treatment | | |
| T48 | | | | | |
| T60 | | 7 pm | | | |
| D3, T72 | 23 | 7 am | * | | |
| | May | | | | |
| D4, T96 | 24 | 7 am | * | | |
| | May | | | | |
| D5, T120 | 25 | 7 am | * | * | |
| | May | | | | |
| Treatment | 20 | 7-8 am | *prior to | * | |
| 1 = D0, T0 | May | | treatment | | |
| T6 | | 1 pm | | | |
| T12 | | 7 pm | | | |
| D1, T24 | | 7 am | * | | |
| Treatment | 22 | 7-8 am | *prior to | * | |
| 2 = D2, | May | | treatment | | |
| T48 | | | | | |
| T60 | | 7 pm | | | |
| D3, T72 | 23 | 7 am | * | | |
| | May | | | | |
| D4, T96 | 24 | 7 am | * | | |
| | May | | | | |
| D5, T120 | 25 | 7 am | * | * | |
| | May | | | | |

Day 0: Treatment Study Start 48 hrs after Prostaglandin injection.
Day 2: Treatment 2
Day 5: T=120 Tissue Collection

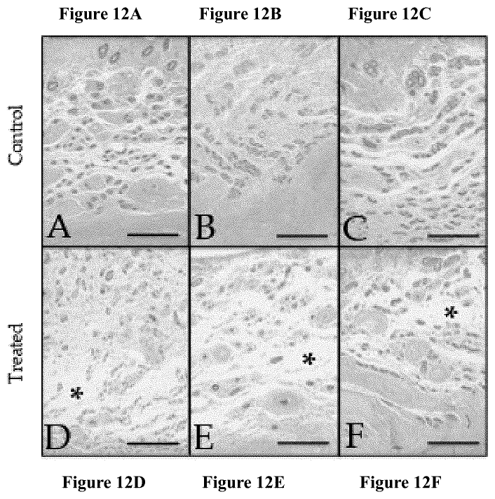

Although relaxin was not measured in the tissues of treated cows in the second experiment, this could have been due to rapid binding of relaxin protein to its receptor, leading to its disappearance from tissues. Importantly, however, histologic changes consistent with the remodeling of connective tissue were seen in the reproductive tracts of the cows treated with mRNA for relaxin, similar to what has been described in females of other species (horses, pigs) that naturally produce relaxin. These changes were not seen in the control cows treated with water alone. Histologic sections of the uterine endometrium from control (FIGS. 12A, 12B, and 12C) and treated (FIGS. 12D, 12E, and 12F) cows demonstrate a moderate increase in clear space (*) within the lamina propria of treated cows. HE, bar=500 μm. These findings are very encouraging with regard to the potential application of relaxin mRNA therapy for the reduction of difficult birth in cattle.

It has been shown that treating cells of the female reproductive tract with messenger RNA (mRNA) coding for relaxin can induce production of relaxin. The mRNA can be modified so the relaxin could be produced for several hours to days, if necessary. Thus, treatment of the female reproductive tract with mRNA for inducing production of relaxin is an advantageous way to help cows, humans, and other female mammals deliver their young in situations where relaxin could help ease delivery.

These data provide evidence in support of the potential use of H2 RLN mRNA therapy as a novel approach in reducing the incidence of dystocia in heifers.

What is claimed is:

1. A synthetic H2 RLN mRNA construct that induces expression of relaxin with a secretion signal, optionally in combination with a reporter protein.

2. The construct of claim 1, wherein the construct induces expression of relaxin when administered to a mammalian kidney.

3. The construct of claim 2, wherein the mammalian kidney is a human kidney or bovine kidney.

4. The construct of claim 1, wherein the construct induces expression of relaxin when administered to mammalian epithelial cells.

5. The construct of claim 4, wherein the mammalian epithelial cells are human epithelial cells or bovine epithelial cells.

6. The construct of claim 1, wherein the reporter protein is NanoLuciferase.

7. The construct of claim 6, wherein the construct is a H2 RLN-NanoLuciferase mRNA construct.

8. The construct of claim 1, wherein the construct is encoded by a nucleotide sequence selected from the group consisting of sequences of SEQ ID NO: 1 and SEQ ID NO: 18.

9. The construct of claim 1, wherein the construct encodes an amino acid sequence selected from the group consisting of sequences of SEQ ID NO: 11 and SEQ ID NO: 23.

10. A method for facilitating delivery of offspring in mammals comprising a step of administering the construct of claim 1 to a mammal prior to delivery of the offspring.

11. The method of claim 10, wherein the mammal is a human or bovine.

12. The method of claim 11, wherein the construct is administered to the kidney.

13. The method of claim 11, wherein the construct is administered to the epithelial cells.

14. The method of claim 12, comprising administration of a dose of 0.5 mg-10 mg of the construct.

15. The method of claim 13, comprising administration of a dose of 0.5 mg-10 mg of the construct.

16. The method of claim 12, wherein two or more doses of 0.5 mg-10 mg of the construct are administered.

17. The method of claim 13, wherein two or more doses of 0.5 mg-10 mg of the construct are administered.
